(19) United States
(12) Patent Application Publication
(10) Pub. No.: US 2007/0083185 A1
(43) Pub. Date: Apr. 12, 2007

(54) IONTOPHORETIC DEVICE AND METHOD OF DELIVERY OF ACTIVE AGENTS TO BIOLOGICAL INTERFACE

(76) Inventor: Darrick Carter, Seattle, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/535,688

(22) Filed: Sep. 27, 2006

Related U.S. Application Data
(60) Provisional application No. 60/722,757, filed on Sep. 30, 2005.

(51) Int. Cl. A61M 31/00

(52) U.S. Cl. 604/501; 604/20

(57) ABSTRACT

An iontophoresis device includes an active electrode element operable to provide an electrical potential; an electrolyte reservoir comprising an electrolyte composition; an inner active agent reservoir comprising a gel matrix and distributed in said gel matrix, a first positively charged active agent; an inner ion selective membrane deposed between said electrolyte reservoir and said inner active agent reservoir; and an outermost ion selective membrane having an outer surface, the outer surface being against the biological interface, wherein, the gel matrix comprises a hydrophilic polycarboxylated polymer having net negative charges.

### CROSS-REFERENCE TO RELATED APPLICATION

This application claims the benefit under 35 U.S.C. § 119(e) of U.S. Provisional Patent Application No. 60/722,757, filed Sep. 30, 2005.

### BACKGROUND OF THE INVENTION

1. Field of the Invention

This disclosure generally relates to the field of iontophoresis, and more particularly to the delivery of active agents such as therapeutic agents or drugs to a biological interface under the influence of electromotive force and/or current.

2. Description of the Related Art

Iontophoresis employs an electromotive force and/or current to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a voltage source, for example a chemical battery. Each electrode assembly typically includes a respective electrode element to apply an electromotive force and/or current. Such electrode elements often comprise a sacrificial element or compound, for example silver or silver chloride.

The active agent may be either cation or anion, and the voltage source can be configured to apply the appropriate voltage polarity based on the polarity of the active agent. Iontophoresis may be advantageously used to enhance or control the delivery rate of the active agent. As discussed in U.S. Pat. No. 5,395,310, the active agent may be stored in a reservoir such as a cavity, or stored in a porous structure or as a gel.

In further development of iontophoresis devices, an ion selective membrane may be positioned to serve as a polarity selective barrier between the active agent reservoir and the biological interface, as discussed in U.S. Pat. No. 5,395,310. The membrane, typically only permeable with respect to one particular type of ions, i.e., that of a charged active agent, prevents the back flux of the oppositely charged ions from the skin or mucous membrane. Although combining membranes in layers with iontophoresis may result in efficient and controlled delivery of the active agents; and allow for flexibility in choosing the electrode system, delivery from these membranes may be difficult. Because the biologically active agent, such as a drug or vaccine, may have to be deposited on the membrane in dry form, the amount of drug absorbed thereon may not be sufficient to meet the dosage requirement.

Dispersing a biologically active agent in a gel matrix may enhance the stability of the agent and the amount of the agent loaded. Of the gel matrices employed in the art, hydrogels are particularly preferred because they inherently contain significant amount of water, which can serve as hydrating reservoirs during iontophoresis. However, the drug loading and release characteristics of hydrogels remain as areas where further investigation is necessary for optimized drug delivery.

Effective and controlled delivery of biologically active agent is an important factor in assessing the commercial acceptance of iontophoresis devices, other factors including cost to manufacture, shelf life or stability during storage, biological capability and/or disposal issues. An iontophoresis device that addresses one or more of these factors is desirable.

### BRIEF SUMMARY OF THE INVENTION

In one embodiment, an iontophoresis device is provided for the delivery of active agents to a biological interface such as skin or mucous membranes that may have improved loading and release characteristics. The device includes an active electrode element operable to provide an electrical potential; an electrolyte reservoir comprising an electrolyte composition; an inner active agent reservoir comprising a gel matrix and distributed in said gel matrix, a first positively charged active agent; wherein, the gel matrix comprises a hydrophilic polycarboxylated polymer having a net negative charge. Optionally, an inner ion selective membrane may be deposed between said electrolyte reservoir and said inner active agent reservoir; and an outermost ion selective membrane having an outer surface, the outer surface being against the biological interface.

In particular, the polycarboxylated polymer gel matrix that binds to a cationic active agent thereby enhances the loading capability. Due to the electrochemically induced pH shift, which causes protons to migrate to the inner active agent reservoir, the negatively charged polycarboxylated polymer gel matrix is neutralized and the cationic active agent dissociate from the gel matrix. In one embodiment, the gel matrix comprises Carbopol™.

In other embodiments, an article of manufacture for transdermal administration of medication by iontophoresis is described, the article of manufacture comprising: an iontophoresis device comprising an active electrode element operable to provide an electrical potential; an electrolyte reservoir comprising an electrolyte composition; and an inner active agent reservoir comprising a gel matrix, the gel matrix including a hydrophilic polycarboxylated polymer having net negative charges; and at least one dosage form comprising one or more active agents, the at least one dosage form loaded in the inner active agent reservoir.

In further embodiments, a method for transdermal administration of an active agent by iontophoresis is described, the method comprising: positioning an active electrode assembly and a counter electrode assembly of an iontophoresis device on a biological interface of a subject, the active electrode assembly further including an active electrode element operable to provide an electrical potential; an electrolyte reservoir comprising an electrolyte composition; and an inner active agent reservoir comprising a gel matrix and distributed in said gel matrix, a positively charged active agent, the gel matrix comprising a hydrophilic polycarboxylated polymer having net negative charges; wherein the positively charged active agent is bound to the gel matrix; and applying a sufficient amount of current to release the active agent from the gel matrix and transport the active agent to the biological interface of the subject, and to administer a therapeutically effective amount of the positively charged active agent in the subject for a limited period of time.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a block diagram of an iontophoresis device comprising active and counter electrode assemblies according to one illustrated embodiment.

FIG. 2 is a block diagram of the iontophoresis device of FIG. 1 positioned on a biological interface, with the outer release liner removed to expose the active agent according to one illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane permits only the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane permits only the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, term “bipolar membrane” means a membrane that is selective to two different charges or polarities. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

Generally speaking, during iontophoresis, charged or uncharged species (including active agents), can migrate across a permeable biological interface into the underlying biological tissue. Typically, an iontophoresis device generates both electro-repulsive and electro-osmotic forces. For charged species, the migration is primarily driven by electro-repulsion between the oppositely charged active electrode and the charged species. In addition to the electro-repulsive forces, the electro-osmotic flow of a liquid (e.g., a solvent) may also contribute to transporting the charged species. In certain embodiments, the electro-osmotic solvent flow is a secondary force that can enhance the migration of the charged species. For uncharged or neutral species, the migration is primarily driven by the electro-osmotic flow of a solvent.

“Active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amine group can typically take the form a quaternary ammonium cation (—NR₃H⁺) at an appropriate pH, also referred to as a protonated amine. As will be discussed in detail below, many active agents, including most of the “caine” class analgesics and anesthetics, comprise amine groups. These amine groups can be present in the iontophoresis device in protonated forms.

The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include Lidocaine®, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

In a preferred embodiment, the active agent described herein, is positively charged or is capable of forming positively charges in aqueous media. These positively charged active agents are also referred to as “cationic active agents”. Many biologically active agents have functional groups that are readily convertible to a positive ion or can dissociate into a positively charged ion and a counter ion in an aqueous medium. Other active agents may comprise polarized or polarizable molecules, which exhibit a polarity at one portion relative to another portion of the molecule. Selections of the suitable active agents are therefore within the knowledge of one skilled in the art. Non-limiting examples of such drugs include Lidocaine®, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes drugs such as exenatide; as well as peptides and proteins for treatment of obesity and other maladies.

As used herein and in the claims, the term “gel matrix” means a type of reservoir, as defined herein, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled polymers or macromolecules (e.g., cylindrical micelles). In other embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

In one embodiment, the gel matrix comprises a biocompatible, hydrophilic polycarboxylated polymer having net negative charges. Typically, the polycarboxylated polymer comprises substantially linear primary polymer particles having carboxylate groups on the polymer backbone, the primary polymer particles being chemically bonded and interconnected via cross-linkers. “Carboxylate group” as used herein refers to carboxylic acid, ester or its salt forms. The linear primary polymer can be generally represented the following formula: 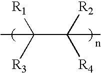
wherein, n is an integer of at least 10. R₁, R₂, R₃ and R₄ are the same or different and independently hydrogen, alkyl, alkenyl, aryl, —COOR or —CH₂COOR, provided at least one of R₁, R₂, R₃ and R₄ is —COOR or —CH₂COOR, wherein R is hydrogen, lower alkyl, aryl, aralkyl, amino (including mono- or di-substituted aminos), or a counter ion.

Alkyl refers to saturated, straight or branched hydrocarbon chains. “Lower alkyl” refers to C₁₋₆ hydrocarbon chain. Examples of alkyls include methyl, ethyl, propyl, butyl, pentyl and hexyl.

Alkenyl refers to hydrocarbon chains having at least one olefinic bond (double bond). Examples of alkenyl include ethenyl, propenyl, butenyl, and the like.

Amino refers to a radical of —NR^{a}R^{b} (wherein, R^{a} and R^{b} are independently hydrogen or alkyl). Examples of aminos include, but are not limited to, —NH2, —N(CH₂CH₃)₂, —NHCH₃, and the like.

Aryl refers to an aromatic cyclic hydrocarbon. An exemplary aryl is phenyl.

Aralkyl refers to an alkyl, in which at least one hydrogen is substituted with an aryl, as defined herein. An example aralkyl is benzyl (i.e., phenylmethyl).

A counter ion, as used herein, refers to a positively charged ion that forms a salt with the carboxylate group. Typically, the counter ion is a physiologically acceptable ion, such as Na⁺, K⁺, NH₄⁺ and the like.

The polymer described herein comprises a plurality of repeating units (monomers). Suitable monomers for preparing polycarboxylated polymers typically include those containing active olefinic bonds. Exemplary monomers include, but are not limited to α,β-unsaturated carboxylic acids, such as acrylic acid, methacrylic acid, ethacrylic acid, α-chloro-acrylic acid, α-cyano acrylic acid, β-methyl-acrylic acid (crotonic acid), α-phenyl acrylic acid, β-acryloxy propionic acid, sorbic acid, α-chloro sorbic acid, angelic acid, cinnamic acid, p-chloro cinnamic acid, β-styryl acrylic acid (1-carboxyl-4-phenyl-1,3-butadiene), itaconic acid, citraconic acid, mesaconic acid, glutaconic acid, aconitic acid, maleic acid, fumaric acid, tricarboxyl ethylene, and the like.

Examples of suitable primary linear polymers having carboxylate groups include but are not limited to: polyacrylic acid, polymethacrylic acid, poly(maleic acid), poly(itaconic acid) and poly(crotonic acid).

For purpose of the present disclosure, polycarboxylated polymer can also be a block copolymer having two or more distinctive blocks of homopolymers, represented by the formula above. Examples of such copolymers include poly(acrylic acid-co-methyl methacrylate), poly(acrylic acid-co-butylacrylate).

In certain embodiments, the polycarboxylated polymers are manufactured by a crosslinking process using one or more monomers described herein, in the presence of cross-linkers. “Cross-linkers” as used herein refers to molecules having multi-functional groups that are capable of forming chemical bonds or crosslinking with monomers of polycarboxylated polymers. Typically, a cross-linker has at least two olefinic bonds. Examples of the cross-linkers include but are not limited to allyl ethers of sucrose, pentaerythritol, polyalkenyl ethers, divinyl glycol, divinylbenzene, N,N-diallylacrylamide, 3,4-dihydroxy-1,5-hexadiene, 2,5-dimethyl-1,5-hexadiene and similar agents.

Typically, the molecular weight of a polycarboxylated polymer is at least 1000. More typically, the molecular weight is at least 5000. Even more typically, the molecular weight is at least 10,000.

In one embodiment, the gel matrix may comprise a carbomer having net negative charges. Carbomers refer to a family of polyacrylic acid cross-linked with polyalkenyl ethers or divinyl glycol. Carbomers typically can swell in water up to 1000 times their original volume to form a gel. The carboxylate groups on the polymer backbone deprotonate when exposed to a pH environment above the pKa of the carboxylate group, which causes repulsion between the negative charges. This repulsion further adds to the swelling of the polymer. “Negatively charged carboxylate group” and “deprotonated carboxylate group” are used herein interchangeably to refer to the COO⁻ group.

Suitable commercial grades of carbomers include Carbopol™ 910, Carbopol™ 934P, Carbopol™ 940, Carbopol™ 941, Carbopol™ 971P, Carbopol™ 974P, Carbopol™ 980, Carbopol™ 981, Carbopol™ 1342, which are well-know rheology modifiers and dermatologic excipients. They are available from B F Goodrich Specialty Chemicals (Cleveland, Ohio). Additional carbomers include Rheogic 252L and Rheogic 250H (both available from Nikon Junyaku) and Hostacerin PN73 (available from Hoechst UK).

The release and loading characteristics of an active agent in a polycarboxylated polymer gel matrix can be functions of a number of factors, including: the degree of the cross-linking of the gel, the pH environment, the density of the negatively charged carboxylate groups.

For instance, carbomers typically have pKa in the range of about 3-6.8. More typically, carbomers have pKa in the range of about 5.5-6.5. While exposed to a pH environment above the pKa, carbomers form a negatively charged gel due to the deprotonated carboxylate groups. Positively charged biologically active agents can bind to the carbomers via ionic interaction. Typically, due to the strong interaction between oppositely charged species, the loading capacity of the negatively charged gel matrix can be optimized for positively charged active agents. This may additionally serve to stabilize the active agent. When local pH is lowered, the negatively charged carboxylate groups are neutralized and the active agents dissociate from the gel matrix in the absence of the ionic interactions.

Advantageously, when an electrical field is applied, the iontophoresis device causes protons to migrate from an electrolyte reservoir to the vicinity of the gel matrix loaded with positively charged active agents. The presence of the protons lowers the local pH of the gel matrix and neutralizes the negatively charged carboxylate groups, thereby causes the dissociation (release) of the active agents. The effect of this electrically-induced pH shift, in combination with the electro-repulsion and/or electro-osmotic forces, promotes the migration and the release of the active agent. This mechanism will be described in more details in connection with the description of the iontophoresis device below.

The active agent can be incorporated into a gel matrix according to the method described in U.S. Pat. No. 6,238,689, which patent is incorporated by reference herein in its entirety. In brief, carbomers are available as fine white powders, which disperse in water to form acidic colloidal suspensions (a 1% dispersion has approx. pH 3) of low viscosity. Neutralization of these suspensions using a base results in the formation of clear translucent gels. Exemplary bases include, without limitation, sodium, potassium or ammonium hydroxides, low molecular weight amines and alkanolamines. An active agent and its salts can form stable hydrophilic complexes with carbomers at about pH 3.5 and are stabilized at an optimal pH of about 5.6.

For example, to prepare an active agent/carbomer complex, the carbomer is suspended in an appropriate solvent, such as water, alcohol or glycerin. Preferably, the carbomer is mixed with water, preferably de-ionized water. Mixtures may range, for example from 0.002 to 0.2 g of carbomer per ml of solvent, preferably from 0.02 to 0.1 g of carbomer per ml of solvent. The mixture is thoroughly mixed at room temperature until a colloidal suspension forms. The mixture may be stirred using a suitable mixer with a blade-type impeller, and the powder sieved into the vortex created by the stirrer using a 500-micron brass sieve. This technique allows ample wetting of the powder and prevents the powder from forming a cluster of particles that may become difficult to wet and disperse.

The active agent or its salt may be diluted with any pharmaceutically acceptable solvent. In a preferred embodiment, the solvent is an alcohol such as ethanol. In another embodiment, the solvent is water. Mixtures may range, for example, from 0.01 to 10 g of drug per ml of solvent, preferably from 0.5 to 5 g of drug per ml solvent. This solution is then added drop wise to the carbomer suspension and mixed continuously until a gel of uniform consistency has formed. Preferably, the active agent/carbomer complex is made by combining 1 g of active agent or its salt with from 0.1 to 100 g of carbomer, more preferably with 1 to 50 g of carbomer. A gradual thickening of the suspension occurs as neutralization of the carbomer takes place. The preparation will now take on the appearance of a slightly white translucent gel.

In one embodiment, the gel matrix loaded with the active agent can be incorporated in its gel form into the inner active agent reservoir, as defined herein. In another embodiment, the gel matrix loaded with the active agent can be dried. According to one embodiment, the gel is vacuum dried. By way of example, the gel is spread on a glass plate and dried under vacuum at 50° C. for about 24 hours. Alternatively, the gel may be freeze-dried. Such methods are well known in the art. The dried powder form can be stored and reconstituted immediately prior to being introduced into the inner active agent reservoir.

The headings provided herein are for convenience only and do not interpret the scope or meaning of the embodiments.

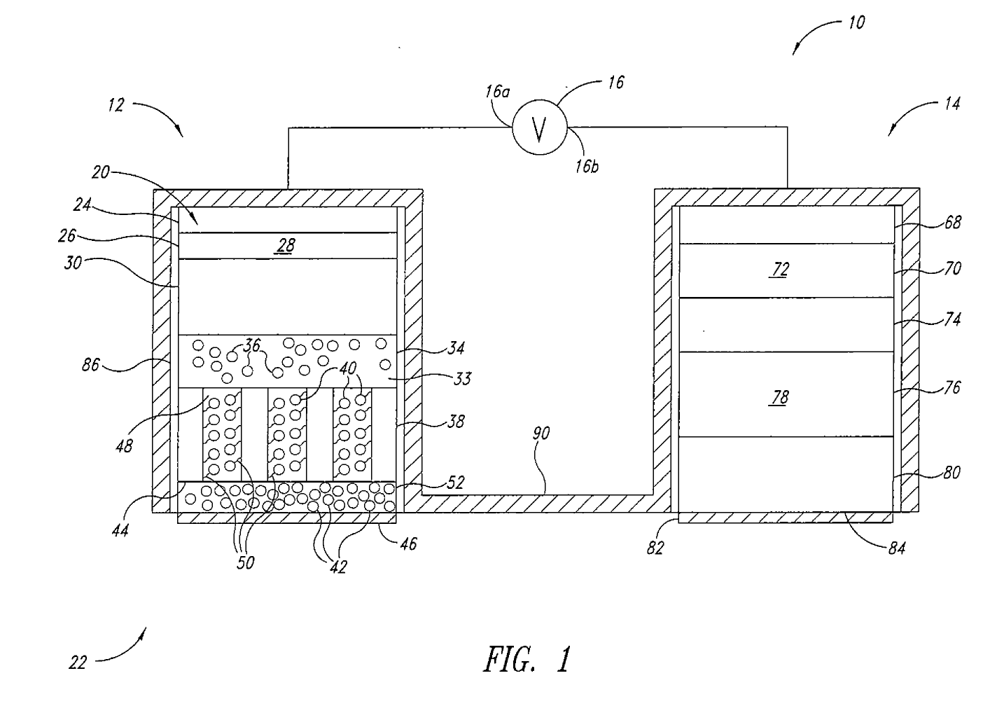

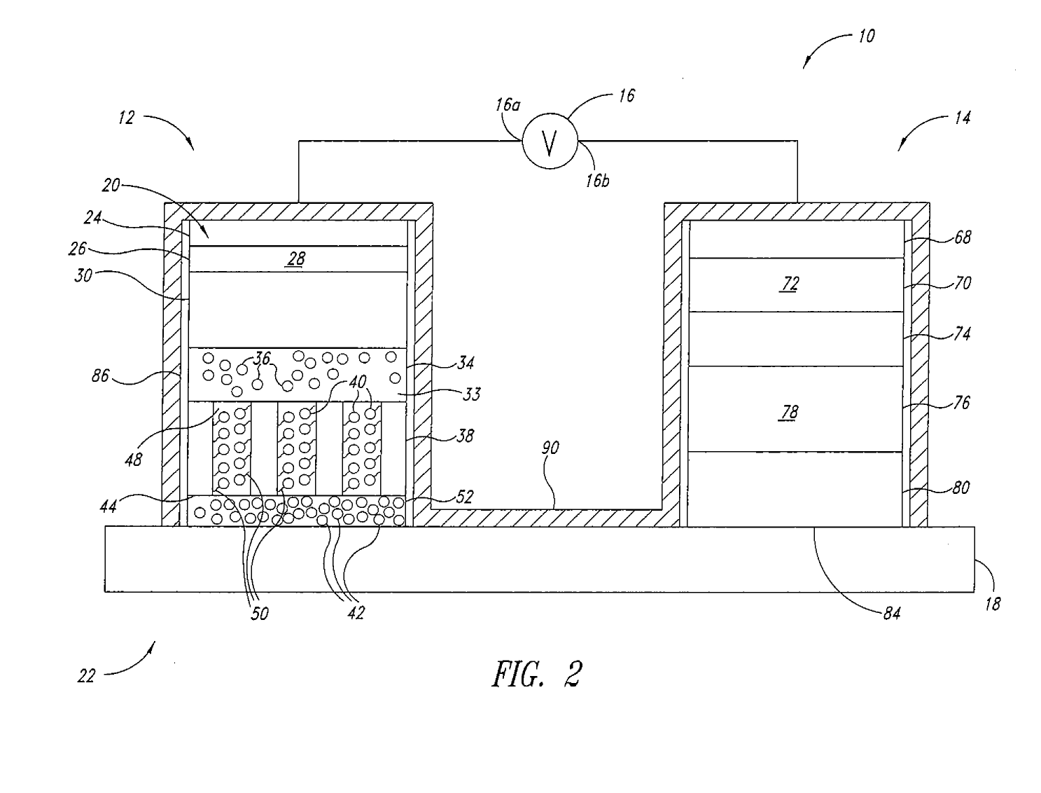

FIGS. 1 and 2 show an iontophoresis device 10 comprising active and counter electrode assemblies, 12, 14, respectively, electrically coupled to a power source 16, operable to supply an active agent contained in the active electrode assembly 12 to a biological interface 18 (FIG. 2), such as a portion of skin or mucous membrane via iontophoresis, according to one illustrated embodiment.

In the illustrated embodiment, the active electrode assembly 12 comprises, from an interior 20 to an exterior 22 of the active electrode assembly 12: an active electrode element 24, an electrolyte reservoir 26 storing an electrolyte 28, an inner ion selective membrane 30, an optional inner sealing liner 32, an inner active agent reservoir 34 storing active agent 36 within a gel matrix 33, an optional outermost ion selective membrane 38 that optionally caches additional active agent 40, an optional further active agent 42 carried by an outer surface 44 of the outermost ion selective membrane 38, and an outer release liner 46. Each of the above elements or structures will be discussed in detail below.

The active electrode element 24 is coupled to a first pole 16a of the power source 16 and positioned in the active electrode assembly 12 to apply an electromotive force or current to transport active agent 36, 40, 42 via various other components of the active electrode assembly 12. When the active agent transported is positively charged, the active electrode element is the anode.

The active electrode element 24 may take a variety of forms. In one embodiment, the device may advantageously employ a carbon-based active electrode element 24. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes because they do not themselves undergo or participate in electrochemical reactions. Thus, an inert electrode distributes current without being eroded or depleted, and conducts current through electrolysis of water, i.e., generating ions (H⁺ and OH⁻) by either oxidation or reduction of water. Additional examples of inert electrodes include stainless steel, gold, platinum or graphite.

Alternatively, an active electrode of a sacrificial conductive material such as a chemical compound or amalgam may also be used. A sacrificial electrode does not cause electrolysis of water, but would itself by oxidized or reduced. Typically for an anode, a metal/metal salt may be employed. In such case, the metal would oxidize to metal ions, which would then be precipitated as an insoluble salt. An example of such anode includes Ag/AgCl electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

In one embodiment, the electrolyte 28 comprises ionic or ionizable components in an aqueous medium, which are able to conduct current towards or away from the active electrode element. Suitable electrolytes include, for example, aqueous solutions of salts. Preferably, the electrolyte 28 includes salts of physiological ions, such as, sodium, potassium, chloride, and phosphate.

Once an electrical potential is applied, water is electrolyzed at both the active and counter electrode assemblies. In the active electrode assembly (i.e., the anode), water is oxidized. As a result, oxygen is removed from water while protons (H⁺) are produced. In one embodiment, the electrolyte 28 may further comprise an anti-oxidant to inhibit the formation of oxygen gas bubbles in order to enhance efficiency and/or increase delivery rates. Examples of biologically compatible anti-oxidants include, but are not limited to ascorbic acid (vitamin C), tocopherol (vitamin E), or sodium citrate.

As noted above, the electrolyte 28 may be in the form of an aqueous solution housed within a reservoir 26, or in the form of dispersion in a hydrogel or hydrophilic polymer capable of retaining a substantial amount of water. For instance, a suitable electrolyte may take the form of a solution of 0.5M disodium fumarate: 0.5M Poly acrylic acid: 0.15M anti-oxidant.

The inner ion selective membrane 30 is generally positioned to separate the electrolyte 28 and the inner active agent reservoir 34. The inner ion selective membrane 30 may take the form of a charge selective membrane. For example, because the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increases the transfer rate and/or biological compatibility of the iontophoresis device 10.

The inner ion selective membrane 30 may further inhibit the transfer of protons (H⁺), however, small amount of protons do permeate across the membrane 30. In certain circumstances, the migration of the protons (H⁺ ions) from the electrolyte reservoir 26 to the inner active agent reservoir 34 may cause potential disadvantages such as reduced efficiency, reduced transfer rate, and/or possible irritation of the biological interface 18 if the proton migration continues toward the biological interface 18. However, for the present device, the proton migration in fact provides a surprising advantage, which will be discussed in further details below.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities or interstices, particularly where the active agent 36 is a liquid.

The inner active agent reservoir 34 further comprises a gel matrix 33, for example, a polycarboxylated polymer gel matrix. As noted above, the polycarboxylated polymer gel matrix is particularly suited for loading large doses of cationic active agents in an active electrode assembly. More specifically, the polycarboxylated polymer gel matrix carries negative charges (COO⁻) at an appropriate pH range, it is therefore capable of coupling with the cationic active agents via strong ionic interaction.

To release the cationic active agents, the gel matrix can be neutralized, which causes the active agents to dissociate. Typically, neutralization occurs when the acidity in the inner active agent reservoir 34 increases. In certain embodiments, during the operation of the iontophoresis device, protons are produced as an electrochemical product of water oxidation in the active electrode assembly 12, for example, an inert electrode. The resulting protons are induced by the electrical current to migrate away from the active electrode assembly 12. As the protons reach the inner active agent reservoir 34, they lower the pH environment of the gel matrix 33 and neutralize the negative charges. The active agents 36 therefore become dissociated from the gel matrix 33 and can be rapidly released. The released active agents migrate toward the biological interface 18 under the electro-repulsion and/or electro-osmotic forces.

Where a sacrificial electrode (e.g., Ag/AgCl) is employed as the anode, the electrode itself is oxidized. Because no proton is produced electrochemically, the electrolyte 28 may be maintained as acidic by a buffer solution. Under the electrical field, a portion of the H⁺ ions in the electrolyte reservoir can be induced to migrate to the inner active agent reservoir 34.

Optionally, an outermost ion selective membrane 38 can be positioned between the inner active agent reservoir 24 and the exterior 22 of the active electrode assembly. The outermost membrane 38 may, as in the embodiment illustrated in FIGS. 1 and 2, take the form of an ion exchange membrane, pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). Under the influence of the electro-repulsion and/or electro-osmotic forces, the ion exchange material or groups 50 substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., Lidocaine®), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent them from competing with one another. Other combinations are possible.

The outer release liner 46 may generally be positioned overlying or covering further active agent 42 carried by the outer surface 44 of the outermost ion selective membrane 38. The outer release liner 46 protects the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2.

An interface-coupling medium (not shown) may be employed between the electrode assembly and the biological interface 18. The interface-coupling medium may, for example, take the form of an adhesive and/or gel. The gel may, for example, take the form of a hydrating gel. Selections of suitable bioadhesive gels are within the knowledge of one skilled in the art.

In the embodiment illustrated in FIGS. 1 and 2, the counter electrode assembly comprises, in an order from an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupled to a second pole 16b of the power source 16, the second pole 16b having an opposite polarity to the first pole 16a. The counter electrode element 68 is therefore the cathode of the device. In one embodiment, the counter electrode element 68 is an inert electrode. For example, the counter electrode element 68 may be the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes are discussed above.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The power source 16 may take the form of one or more chemical battery cells, super- or ultra-capacitors, or fuel cells. The power source 16 may be selectively electrically coupled to the active and counter electrode assemblies 12, 14 via a control circuit (not shown), which may include discrete and/or integrated circuit elements to control the voltage, current and/or power delivered to the electrode assemblies 12 and 14.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

Thus, certain embodiments describe an article of manufacture for transdermal administration of medication by iontophoresis, comprising: an iontophoresis device comprising an active electrode element operable to provide an electrical potential; an electrolyte reservoir comprising an electrolyte composition; and an inner active agent reservoir including a gel matrix, the gel matrix being a hydrophilic polycarboxylated polymer having net negative charges; and at least one dosage form comprising one or more active agents, the at least one dosage form loaded in the inner active agent reservoir.

Further, certain embodiments describe a method for transdermal administration of an active agent by iontophoresis, comprising: positioning an active electrode assembly and a counter electrode assembly of an iontophoresis device on a biological interface of a subject, the active electrode assembly further including an active electrode element operable to provide an electrical potential; an electrolyte reservoir comprising an electrolyte composition; and an inner active agent reservoir comprising a gel matrix and distributed in said gel matrix, a positively charged active agent, the gel matrix comprising a hydrophilic polycarboxylated polymer having net negative charges; wherein the positively charged active agent is bound to the gel matrix; and applying a sufficient amount of current to release the active agent from the gel matrix and transport the active agent to the biological interface of the subject, and to administer a therapeutically effective amount of the positively charged active agent in the subject for a limited period of time.

In certain embodiments, when in use, the iontophoresis device produces a small quantity of protons, which migrate from the electrolyte reservoir 26 across the inner ion (anion) selective membrane 30 to the inner active agent reservoir 34. The presence of the protons neutralizes the negatively charged gel matrix 33 and causes the cationic active agent 36 to dissociate from gel matrix and transport toward the biological interface 18. Optionally, additional active agent 40 is released by the ion exchange groups or material 50 by the substitution of ions of the same charge or polarity (e.g., active agent 36), and transported toward the biological interface 18. While some of the active agent 36 may substitute for the additional active agent 40, some of the active agent 36 may be transferred through the outermost ion elective membrane 38 into the biological interface 18. Further optional active agent 42 carried by the outer surface 44 of the outermost ion elective membrane 38 is also transferred to the biological interface 18.

In use, the outermost ion selective membrane 38 may be placed directly in contact with the biological interface 18. Alternatively, an interface-coupling medium (not shown) may be employed between the outermost ion selective membrane 38 and the biological interface 18. The interface-coupling medium may, for example, take the form of an adhesive and/or gel. The gel may, for example, take the form of a hydrating gel or a hydrogel. If used, the interface-coupling medium should be permeable by the active agent 34.

The power source 16 may take the form of one or more chemical battery cells, super- or ultra-capacitors, or fuel cells. The power source 16 may, for example, provide a voltage of 12.8V DC, with tolerance of 0.8V DC, and a current of 0.3 mA. The power source 16 may be selectively electrically coupled to the active and counter electrode assemblies 12, 14 via a control circuit, for example, via carbon fiber ribbons. The iontophoresis device 10a may include discrete and/or integrated circuit elements to control the voltage, current and/or power delivered to the electrode assemblies 12, 14. For example, the iontophoresis device 10 may include a diode to provide a constant current to the electrode elements 20, 40.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiment may include a control circuit or subsystem to control a voltage, current or power applied to the active and counter electrode elements 20, 40. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The iontophoresis device discussed above may advantageously be combined with other microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non- biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the compounds and drugs may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight compounds and drugs, such as those comprising proteins, peptides and/or nucleic acids, and corresponding compositions thereof. In certain embodiments, for example wherein the fluid is an ionic solution, microneedle(s) or microneedle array(s) can provide electrical continuity between a voltage source and the tip of the microneedle(s). Microneedle(s) or microneedle array(s) may be used advantageously to deliver or sample compounds or compositions by iontophoretic methods, as disclosed herein.

Accordingly, in certain embodiments, for example, a plurality of microneedles in an array may advantageously be formed on an outermost biological interface-contacting the outer surface of an iontophoresis device. Active agents delivered or sample by such a device may comprise, for example, high-molecular weight molecules or drugs, such as proteins, peptides and/or nucleic acids.

In certain embodiments, compounds or compositions can be delivered by an iontophoresis device comprising an active electrode assembly and a counter electrode assembly, electrically coupled to a voltage source to deliver an active agent to, into, or through a biological interface. The active electrode assembly includes the following: a first electrode member connected to a positive electrode of the voltage source; a active agent reservoir having a drug solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a voltage source to deliver an active agent to, into, or through a biological interface. The active electrode assembly includes the following: a first electrode member connected to a positive electrode of the voltage source; a first electrolyte holding part having an electrolyte that is in contact with the first electrode member and to which is applied a voltage via the first electrode member; a first anion-exchange membrane that is placed on the forward surface of the first electrolyte holding part; a active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte holding part; a third electrolyte holding part that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte holding part; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488970, filed Mar. 9, 2004; U.S. provisional application Ser. No. 60/722,757, filed Sep. 30, 2005; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

Aspects of the various embodiments can be modified, if necessary, to employ systems, circuits and concepts of the various patents, applications and publications to provide yet further embodiments.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

I claim:

1. An iontophoresis device to delivery active agents to a biological interface, the iontophoresis device comprising an active electrode assembly and a counter electrode assembly, the active electrode assembly further including:
an active electrode element operable to provide an electrical potential;
an electrolyte reservoir comprising an electrolyte composition; and
an inner active agent reservoir including a gel matrix and distributed in said gel matrix, a first positively charged active agent, the gel matrix comprising a hydrophilic polycarboxylated polymer having net negative charges.

2. The iontophoresis device of claim 1 wherein the polycarboxylated polymer comprises linear primary polymer particles having a plurality of carboxylate groups, the primary polymer particles being chemically bonded and interconnected via cross-linkers.

3. The iontophoresis device of claim 2 wherein the linear primary polymer is represented by the following formula: 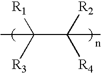
wherein,
n is an integer of at least 10;
R₁, R₂, R₃ and R₄ are the same or different and independently hydrogen, alkyl, alkenyl, —COOR or —CH₂COOR, provided at least one of R₁, R₂, R₃ and R₄ is —COOR or —CH₂COOR, and
R is hydrogen, lower alkyl, aryl, aralkyl, amino (including mono- or di-substituted aminos), or a counter ion.

4. The iontophoresis device of claim 2 wherein the cross-linker is a molecule comprising at least two olefinic bonds.

5. The iontophoresis device of claim 4 wherein the cross-linker is allyl ethers of sucrose, pentaerythritol, polyalkenyl ethers, divinyl glycol, divinylbenzene, N,N-diallylacrylamide, 3,4-dihydroxy-1,5-hexadiene, 2,5-dimethyl-1,5-hexadiene and similar agents.

6. The iontophoresis device of claim 2 wherein the polycarboxylated polymer is a Carbopol™.

7. The iontophoresis device of claim 1 wherein the electrolyte composition comprises water, physiological ions and an anti-oxidant.

8. The iontophoresis device of claim 7 wherein the electrolyte composition includes a hydrogel.

9. The iontophoresis device of claim 7 wherein the anti-oxidant is ascorbic acid, tocopherol, sodium citrate or a combination thereof.

10. The iontophoresis device of claim 2 wherein the positively charged active agents bind to the negatively charged polycarboxylated polymer gel matrix via ionic interaction at pH in the range of about 3-6.8.

11. The iontophoresis device of claim 10 wherein the positively charged active agents dissociate from polycarboxylated polymer gel matrix at pH below 3.

12. The iontophoresis device of claim 2 wherein the positively charged active agents bind to the negatively charged polycarboxylated polymer gel matrix via ionic interaction at pH in the range of about 5.5-6.5.

13. The iontophoresis device of claim 12 wherein the positively charged active agents dissociate from polycarboxylated polymer gel matrix at pH below 5.5.

14. The iontophoresis device of claim 1 further comprising an inner ion selective membrane positioned between said electrolyte reservoir and said inner active agent reservoir,

15. The iontophoresis device of claim 14 wherein the inner ion selective membrane substantially passes anions and substantially blocks cations.

16. The iontophoresis device of claim 14 further comprising an outermost ion selective membrane having an outer surface, the outer surface being proximate the biological interface when in use.

17. The iontophoresis device of claim 16 the outer ion selective membrane substantially passes cations and substantially blocks anion.

18. The iontophoresis device of claim 16 further comprising a second positively charged active agent cached in the outermost ion selective membrane.

19. The iontophoresis device of claim 16 further comprising a third positively charged active agent deposited on the outer surface of the outermost ion selective membrane.

20. The iontophoresis device of claim 16 further comprising an outer release liner underlying said outer surface, said outer release liner being proximate the biological interface when in use.

21. The iontophoresis device of claim 1 further comprising a microneedle array contacting an outer surface of the iontophoresis device.

22. The iontophoresis device of claim 1 wherein the active electrode element is an inert electrode.

23. The iontophoresis device of claim 22 wherein the electrolyte composition comprising water and protons are produced when in use.

24. The iontophoresis device of claim 1 wherein the positively charged active agents are centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, marcaine, chloroprocaine, etidocaine, prilocaine, lignocaine, benzocaine, zolamine, ropivacaine, and dibucaine, or combinations thereof.

25. The iontophoresis device of claim 1 wherein the first positively charged active agent is Lidocaine®.

26. An article of manufacture for transdermal administration of medication by iontophoresis, comprising:
an active electrode assembly including an active electrode element operable to provide an electrical potential; an electrolyte reservoir comprising an electrolyte composition; and an inner active agent reservoir including a gel matrix, the gel matrix being a hydrophilic polycarboxylated polymer having net negative charges; and
at least one dosage form comprising one or more active agents loaded in the inner active agent reservoir.

27. The article of manufacture of claim 26 wherein the one or more active agents are positively charged.

28. The article of manufacture of claim 26 wherein the one or more active agents include analgesic or anesthetic agents.

29. The article of manufacture of claim 28 wherein the one or more active agents are centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, methyl chloride, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, marcaine, chloroprocaine, etidocaine, prilocaine, lignocaine, benzocaine, zolamine, ropivacaine, and dibucaine, or combinations thereof.

30. The article of manufacture of claim 28 wherein the one or more active agents include Lidocaine®.

31. The article of manufacture of claim 27 wherein the iontophoresis device further comprises an inner ion selective membrane positioned between the electrolyte reservoir and the inner active agent reservoir.

32. The article of manufacture of claim 31 wherein the inner ion selective membrane passes anions and substantially blocks cations.

33. The article of manufacture of claim 31 wherein the iontophoresis device further comprises an outer ion selective membrane, wherein the outer ion selective membrane passes cations and substantially blocks anions.

34. The article of manufacture of claim 26 further comprising a counter electrode assembly.

35. A method for transdermal administration of an active agent by iontophoresis, comprising:
positioning an active electrode assembly and a counter electrode assembly of an iontophoresis device on a biological interface of a subject, the active electrode assembly including an active electrode element operable to provide an electrical potential; an electrolyte reservoir comprising an electrolyte composition; and an inner active agent reservoir comprising a gel matrix and distributed in said gel matrix, a positively charged active agent, the gel matrix comprising a hydrophilic polycarboxylated polymer having net negative charges; wherein the positively charged active agent is bound to the gel matrix; and
applying a sufficient amount of current to release the active agent from the gel matrix and transport the active agent to the biological interface of the subject, and to administer a therapeutically effective amount of the positively charged active agent in the subject for a limited period of time.

36. The method of claim 35 wherein applying the sufficient amount of current include electrochemically oxidizing water in the electrolyte composition and producing protons.

37. The method of claim 36 wherein the protons neutralize the negative charge of the gel matrix and cause the positively charged active agent to be released.
